(19) United States
(12) Patent Application Publication
(10) Pub. No.: US 2021/0114236 A1
(43) Pub. Date: Apr. 22, 2021

(54) SURGICAL ROBOTIC SYSTEMS

(71) Applicant: Covidien LP, Mansfield, MA (US)

(72) Inventor: Jaimeen Kapadia, Cambridge, MA (US)

(21) Appl. No.: 17/047,483

(22) PCT Filed: Mar. 28, 2019
(86) PCT No.: PCT/US19/24523

§ 371 Date: Oct. 14, 2020

Related U.S. Application Data
(60) Provisional application No. 62/658,148, filed on Apr. 16, 2018.

(51) Int. Cl. B25J 18/02; A61B 34/30; A61B 17/00; B25J 9/10

(52) U.S. Cl. CPC B25J 18/025; A61B 34/30; A61B 2090/506; B25J 9/104; A61B 17/00

(57) ABSTRACT

A surgical robotic system includes an elongated slide and a carriage for supporting an instrument drive unit. The slide includes an inner shaft and an outer sleeve disposed about the inner shaft. The outer sleeve is configured to move relative to the inner shaft along a longitudinal axis defined by the slide between a retracted position, in which the slide has a first length, and an extended position, in which the slide has a second length, greater than the first length. The carriage is coupled to the outer sleeve and movable relative to the outer sleeve along the longitudinal axis.

### BACKGROUND

Surgical robotic systems have been used in minimally invasive medical procedures. Some surgical robotic systems included a console supporting a surgical robotic arm and a surgical instrument having at least one end effector (e.g., forceps or a grasping tool) mounted to the robotic arm. The robotic arm provided mechanical power to the surgical instrument for its operation and movement.

Manually-operated surgical instruments often included a handle assembly for actuating the functions of the surgical instrument. However, when using a robotic surgical system, no handle assembly was typically present to actuate the functions of the end effector. Accordingly, to use each unique surgical instrument with a robotic surgical system, an instrument drive unit was used to interface with the selected surgical instrument to drive operations of the surgical instrument.

The instrument drive unit was typically coupled to the robotic arm via a slide. The slide allowed the instrument drive unit and the attached surgical instrument to move along an axis of the slide, providing a means for adjusting the axial position of the end effector of the surgical instrument relative to a patient.

### SUMMARY

In accordance with an aspect of the present disclosure, a surgical robotic system is provided and includes an elongated slide and a carriage for supporting an instrument drive unit. The slide defines a longitudinal axis and includes an inner shaft, and an outer sleeve disposed about the inner shaft. The outer sleeve is configured to move relative to the inner shaft along the longitudinal axis between a retracted position, in which the slide has a first length, and an extended position, in which the slide has a second length, greater than the first length. The carriage is coupled to the outer sleeve and movable relative to the outer sleeve along the longitudinal axis.

In aspects, the carriage may be movable along the outer sleeve between a descended position, in which the carriage is disposed adjacent a bottom end portion of the outer sleeve, and an ascended position, in which the carriage is disposed adjacent a top end portion of the outer sleeve.

In aspects, the top end portion of the outer sleeve may have a surface feature and the carriage may be configured to engage the surface feature upon moving to the ascended position, such that further ascension of the carriage effects a movement of the outer sleeve toward the extended position.

In aspects, the outer sleeve may be configured to move toward the retracted position simultaneously with movement of the carriage toward the descended position.

In aspects, the top end portion of the outer sleeve may have a lock configured to selectively fix the carriage to the top end portion, such that a downward force exerted on the carriage is transferred to the outer sleeve to move the outer sleeve relative to the inner shaft from the extended position toward the retracted position.

In aspects, the lock may be configured to release the carriage upon the outer sleeve entering the retracted position, such that a further downward force exerted on the carriage causes the carriage to descend along the outer sleeve.

In aspects, the surgical robotic system may further include a belt and pulley system operably coupled to the carriage for moving the carriage along the outer sleeve.

In aspects, the belt and pulley system may include a pair of first and second pulleys coupled to the outer sleeve, a third pulley coupled to the inner shaft, and a belt operably coupled to each of the first, second and third pulleys. The carriage may be fixed to the belt, such that movement of the belt drives a movement of the carriage along the outer sleeve.

In aspects, the surgical robotic system may further include a motor operably coupled to the belt for driving the movement of the belt.

In aspects, the first pulley may be axially fixed to a top end portion of the outer sleeve, the second pulley may be axially fixed to a bottom end portion of the outer sleeve, and the third pulley may be axially fixed to a top end portion of the inner shaft.

In aspects, the second pulley may be disposed between the first and third pulleys. The first pulley may be disposed a first distance from a vertical axis that extends through a center of the second pulley and parallel with the longitudinal axis of the slide. The third pulley may be disposed a second distance from the vertical axis. The second distance may be less than the first distance.

In aspects, the first and third pulleys may be disposed adjacent one another when the outer sleeve is in the retracted position, and longitudinally spaced from one another when the sleeve is in the extended position.

In aspects, the surgical robotic system may further include a robotic arm having the inner shaft coupled thereto.

Further details and aspects of exemplary embodiments of the present disclosure are described in more detail below with reference to the appended figures.

As used herein, the terms parallel and perpendicular are understood to include relative configurations that are substantially parallel and substantially perpendicular up to about + or −10 degrees from true parallel and true perpendicular.

### BRIEF DESCRIPTION OF THE DRAWINGS

Embodiments of the present disclosure are described herein with reference to the accompanying drawings, wherein:

FIG. 1 is a schematic illustration of a surgical robotic system including an instrument drive unit coupled to a slide in accordance with the present disclosure;

FIG. 2 is a front view of the instrument drive unit and an associated surgical instrument coupled to an exemplary embodiment of a slide;

FIG. 3 is a side view, with parts removed, of a carriage coupled to the slide of FIG. 2;

FIG. 4 is a front view, with parts removed, of the carriage coupled to the slide;

FIG. 5 is a side view of the slide, with an outer shaft of the slide shown in phantom, illustrating an inner shaft of the slide;

FIG. 6 is a side view of the slide, illustrating a belt and pulley system of the surgical robotic system;

FIG. 7 is a side perspective view, with parts removed, of the carriage coupled to the slide, illustrating the slide in an extended configuration and the carriage in an ascended position;

FIG. 8 is a perspective view of another embodiment of a slide for use in the surgical robotic system of FIG. 1, illustrating the slide in a retracted configuration;

FIG. 9 is rear perspective view of the slide of FIG. 8 illustrated in the extended configuration; and

FIG. 10 is a front perspective view of the slide of FIG. 8 illustrated in the extended configuration.

### DETAILED DESCRIPTION

Embodiments of the presently disclosed surgical robotic system and methods of use thereof are described in detail with reference to the drawings, in which like reference numerals designate identical or corresponding elements in each of the several views. As used herein, the term “distal” refers to that portion of the surgical robotic system or component thereof that is closest to the patient, while the term “proximal” refers to that portion of the surgical robotic system or component thereof further from the patient.

As will be described in detail below, provided is a surgical robotic system including a robotic arm, an elongated slide or rail coupled to the robotic arm, and an instrument drive unit configured to drive an operation of an attached surgical instrument. The slide is constructed from an inner shaft and an outer sleeve that is disposed over the inner shaft and slidable relative to the inner shaft between retracted and extended positions. The outer sleeve has a track along which a carriage, that supports the instrument drive unit, moves between descended and ascended positions. The sleeve may include a belt and pulley system that functions to move both the carriage relative to the outer sleeve, and the outer sleeve relative to the inner shaft.

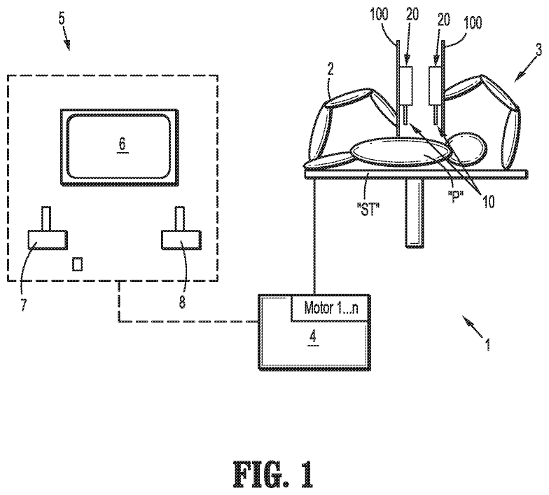

Referring initially to FIG. 1, a surgical system, such as, for example, a surgical robotic system 1, generally includes a plurality of surgical robotic arms 2, 3 having an instrument drive unit 20 and an electromechanical instrument 10 removably attached thereto; a control device 4; and an operating console 5 coupled with control device 4. Operating console 5 includes a display device 6, which is set up in particular to display three-dimensional images; and manual input devices 7, 8, by means of which a person (not shown), for example a surgeon, is able to telemanipulate robotic arms 2, 3 in a first operating mode, as known in principle to a person skilled in the art.

Each of the robotic arms 2, 3 may be composed of a plurality of members, which are connected through joints. Robotic arms 2, 3 may be driven by electric drives (not shown) that are connected to control device 4. Control device 4 (e.g., a computer) is set up to activate the drives, in particular by means of a computer program, in such a way that robotic arms 2, 3, the attached instrument drive units 20, and thus electromechanical instrument 10 execute a desired movement according to a movement defined by means of manual input devices 7, 8. Control device 4 may also be set up in such a way that it regulates the movement of robotic arms 2, 3 and/or of the drives.

Surgical robotic system 1 is configured for use on a patient “P” lying on a surgical table “ST” to be treated in a minimally invasive manner by means of a surgical instrument, e.g., electromechanical instrument 10. Surgical robotic system 1 may also include more than two robotic arms 2, 3, the additional robotic arms likewise being connected to control device 4 and being telemanipulatable by means of operating console 5. A surgical instrument, for example, an electromechanical surgical instrument 10 (including an electromechanical end effector (not shown)), may also be attached to the additional robotic arm.

Control device 4 may control a plurality of motors, e.g., motors (Motor 1 . . . n), with each motor configured to drive movement of robotic arms 2, 3 in a plurality of directions. Further, control device 4 may control a plurality of motors (not explicitly shown) of instrument drive unit 20 to drive various operations of surgical instrument 10. The instrument drive unit 20 transfers power and actuation forces from its motors to driven members (not shown) of the electromechanical instrument 10 to ultimately drive movement of components of the end effector (not shown) of the electromechanical instrument 10, for example, a movement of a knife blade (not shown) and/or a closing and opening of jaw members (not shown) of the end effector.

For a more detailed description of the construction and operation of components of an exemplary robotic surgical system, reference may be made to U.S. Pat. No. 8,828,023, entitled “Medical Workstation,” (hereinafter, “the '023 Patent”), and International Patent Publication WO2017/205308A1, entitled “Robotic Surgical Assemblies,” filed on May 23, 2017, (hereinafter, “the '308 Publication”), the entire contents of each of which are incorporated by reference herein.

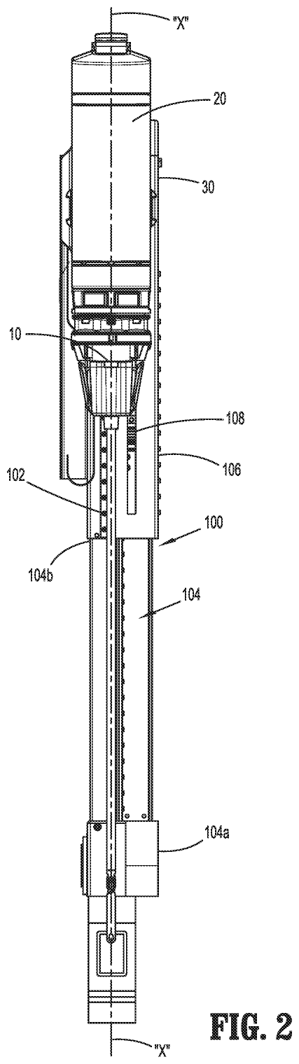

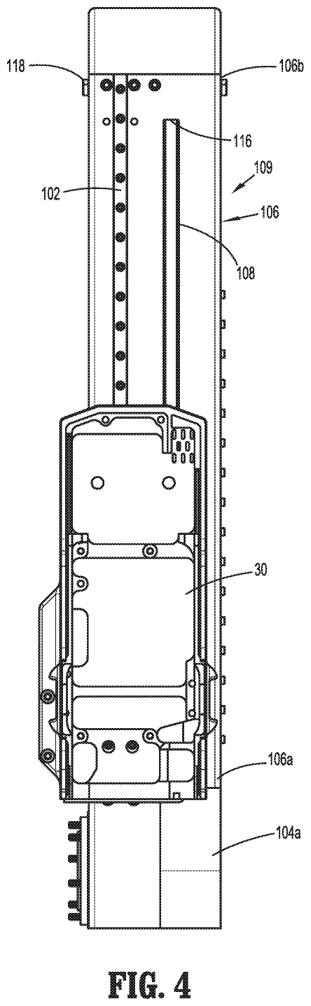

With reference to FIGS. 2-7, the surgical robotic system 1 includes a carriage 30 on which the instrument drive unit 20 is supported or carried, and the slide 100, which supports the carriage 30. The carriage 30 is configured to fix the instrument drive unit 20 thereto, such that movement of the carriage 30 along and relative to the slide 100 causes the instrument drive unit 20 to move therewith. The carriage 30 is slidably coupled to a linear track 102 defined longitudinally along an outer sleeve 106 of the slide 100, as will be described below.

The slide 100 may have a generally rectangular shape and is constructed from an inner shaft 104 and an outer sleeve or sheath 106 disposed around the inner shaft 104. In embodiments, the slide 100 may assume any suitable shape, such as, for example, tubular or cylindrical. The inner shaft 104 is coupled to an end of the robotic arm 2 (FIG. 1) either in a fixed or rotatable manner. The inner shaft 104 has a bottom end portion 140a and a top end portion 104b and defines a longitudinal axis “X” therebetween. The inner shaft 104 may have an overall length approximately equal to half the length of a conventional slide.

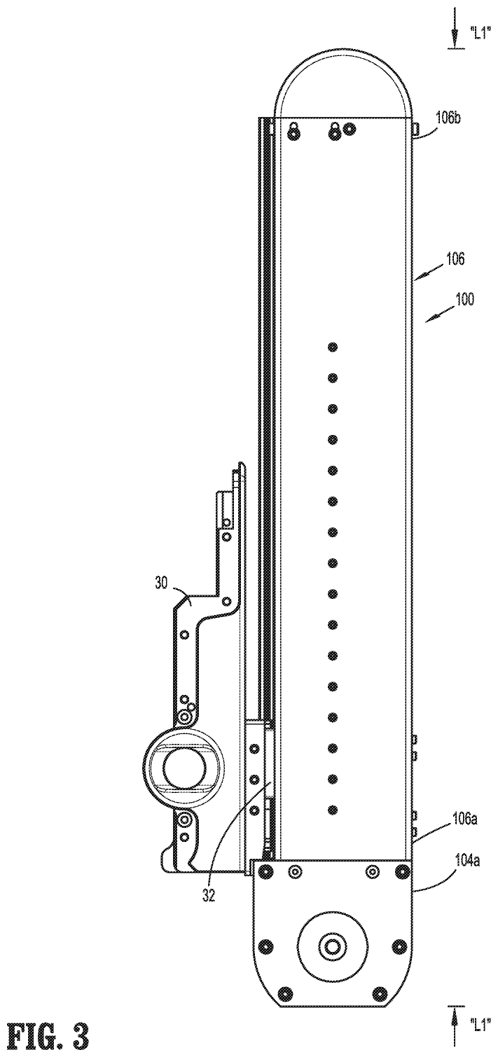

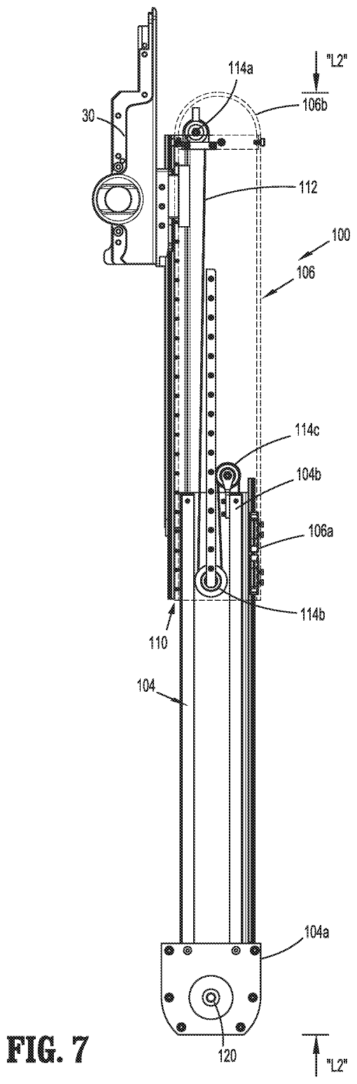

The outer sleeve 106 of the slide 100 is disposed about the inner shaft 104 and is telescopically coupled thereto. As such, the outer sleeve 106 is slidable along and relative to the longitudinal axis “X” of the inner shaft 104 between a retracted position, as shown in FIG. 3, and an extended position, as shown in FIG. 7. When the outer sleeve 106 is in the retracted position, the slide has a first length “L1” (FIG. 3), substantially equal to approximately half the length of a conventional slide (e.g., as shown and described in the '023 Patent, and the '308 Publication), and when the outer sleeve is in the extended position, the slide 100 has a second length “L2” (FIG. 7), substantially equal to approximately the full length of a conventional slide.

The outer sleeve 106 of the slide 100 defines a longitudinally-extending track 102. The track 102 of the outer sleeve 106 may be a single rail or a pair of parallel rails. As mentioned above, the carriage 30 is slidably coupled to the track 102 of the outer sleeve 106. More specifically, the carriage 30 has a coupling member or flange 32 extending from a back side thereof and through an elongated slot 108 of the outer sleeve 106. The coupling member 32 of the carriage 30 is received in an interior chamber 110 (FIG. 7) of the outer sleeve 106 and is fixed to a belt or cable 112 of a belt and pulley system 114 of the slide 100 for driving the movement of the carriage 30 between the ascended and descended positions, as will be described in detail.

The elongated slot 108 is defined along the length of the outer sleeve 106 and runs parallel with the track 102 between a bottom end portion 106a of the outer sleeve 106 and a top end portion 106b of the outer sleeve 106. The elongated slot 108 of the outer sleeve 106 has an upper limit defining a surface feature 116 that prevents the carriage 30 from ascending beyond the upper limit. In embodiments, the surface feature 116 may be a projection extending outwardly from the top end portion 106b of the outer sleeve 106. Upon the coupling member 32 of the carriage 30 contacting the surface feature 116, a threshold force exerted on the carriage 30 in an upward direction causes the outer sleeve 106 to rise relative to the inner shaft 104.

The top end portion 106b may further include a locking feature 118, such as, for example, a roller catch, a magnetic latch, or the like. The locking feature 118 is configured to selectively lock the carriage 30 to the top end portion 106b of the outer sleeve 106 when the carriage 30 enters the ascended position. As such, with the outer sleeve 106 in the extended position relative to the inner shaft 104, as shown in FIG. 7, a downward force exerted on the carriage 30 via the belt 112 causes the outer sleeve 106 to move downwardly with the carriage 30 due to the locking feature 118 locking the outer sleeve 106 and the carriage 30 to one another. Upon the bottom end portion 106a of the outer sleeve 106 bottoming out on the bottom end portion 104a of the inner shaft 104, the locking feature 118 releases the carriage 30 to allow the carriage 30 to descend along the track 102 of the outer sleeve 106.

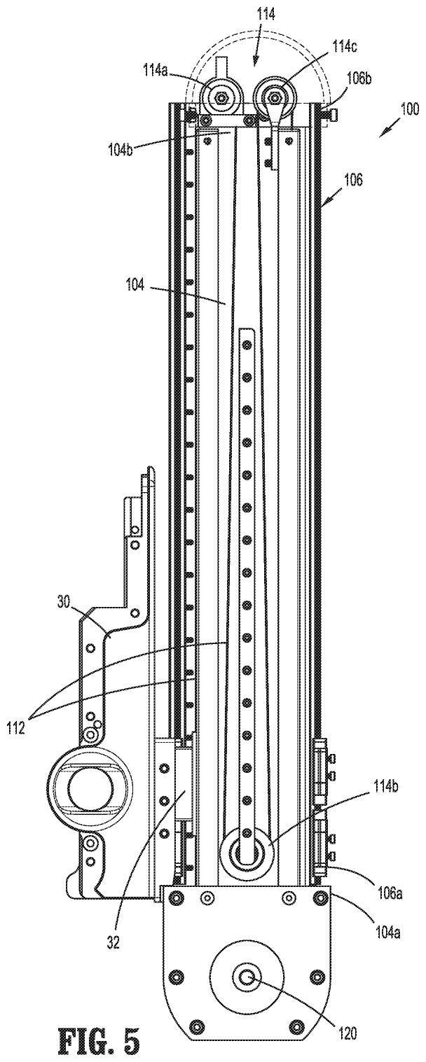

With reference to FIGS. 5-7, the belt and pulley system 114 or drivetrain of the slide 100 is illustrated. The drivetrain 114 is operably coupled to a drive motor 120 disposed in the bottom end portion 104a of the inner shaft 104. The drivetrain 114 includes a pair of first and second pulleys 114a, 114b coupled to the outer shaft 106, and a third pulley 114c coupled to the inner shaft 104. The first pulley 114a is axially fixed and rotatably coupled to the top end portion 106b of the outer sleeve 106 of the slide 100, and the second pulley 114b is axially fixed and rotatably coupled to the bottom end portion 10ba of the outer sleeve 106. As such, as the outer sleeve 106 moves relative to the inner shaft 104 toward the extended position, the first and second pulleys 114a, 114b move therewith. The third pulley 114c is axially fixed and rotatably coupled to the top end portion 104b of the inner shaft 104.

The second pulley 114b is disposed between the first and third pulleys 114a, 114c and is longitudinally spaced from the first pulley 114a along the length of the outer sleeve 106. As shown in FIG. 5, when the outer sleeve 106 is in the retracted position, the first and third pulleys 114a, 114c are disposed adjacent one another, with the second pulley 114b longitudinally spaced from the third pulley 114c. As shown in FIG. 7, when the outer sleeve 106 is in the extended position, the first and third pulleys 114a, 114c are longitudinally spaced from one another, with the second and third pulleys 114b, 114c proximate to one another.

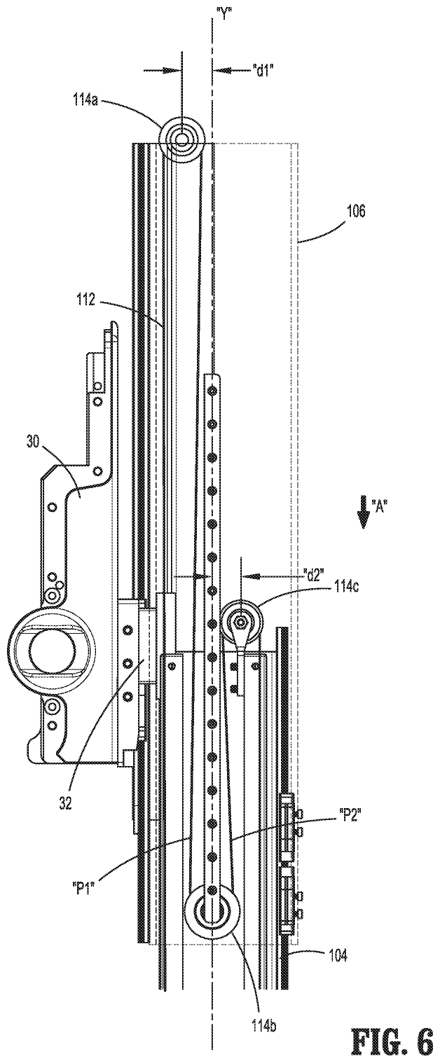

With specific reference to FIG. 6, the second pulley 114b is positioned relative to the first and third pulleys 114a, 114c so that a net downward force, in the direction indicated by arrow “A” in FIG. 6, is exerted on the outer sleeve 106. In particular, the second pulley 114b has a vertical axis “Y” extending through a center point thereof and parallel with the longitudinal axis “X” (FIG. 2) of the inner shaft 104. The first pulley 114a is disposed a first distance “d1” from the vertical axis “Y” in a transverse direction, and the third pulley 114c is disposed a second distance “d2” from the vertical axis “Y” in the transverse direction, less than the first distance “d1.” Accordingly, a first portion “P1” of the belt 112 extends from the second pulley 114b to the first pulley 114a at an angle greater than an angle at which a second portion “P2” of the belt 112 extends from the second pulley 114b to the third pulley 114c. Due to the difference in these angles, the downward force exerted by the first pulley 114a on the outer sleeve 106 is greater than the upward force exerted by the third pulley 114c on the outer sleeve 106, whereby the outer sleeve 106 has a constant net downward force imparted thereon. Stated differently, a sum of all of the Y-components of force acting on first portion “P1” of belt 112, and second portion “P2” of belt 112, due to the angles of inclination of first portion “P1” and second portion “P2” of belt 112, is such that there is constant net downward force imparted on outer sleeve 106.

The belt 112 is operably coupled to the drive motor 120 and each of the pulleys 114a-c. The belt 112 is wrapped over the first pulley 114a, under the second pulley 114b, and over the third pulley 114c. The belt 112 is driven by the motor 120 and is fixed to the coupling member 32 of the carriage 30, such that an activation of the motor 120 causes the belt 112 to move around the pulleys 114a-c and move the attached carriage 30 along the outer sleeve 106 either toward the ascended position or the descended position.

In operation, prior to performing a surgical procedure, the instrument dive unit 20 may be attached to the carriage 30, and the electromechanical instrument 10 may be attached to the instrument drive unit 20. With the instrument drive unit 20 and the associated electromechanical instrument 10 attached to the carriage 30, a longitudinal position (e.g., height) of the carriage 30 along the longitudinal axis “X” may be adjusted. For example, to raise the carriage 30, the motor 120 of the slide 100 is activated to move the belt 112 upwardly relative to the outer sleeve 106 of the slide 100. The carriage 30 is raised to the ascended position and contacts the locking feature 118 and/or the surface feature 116 of the top end portion 106b of the outer sleeve 106. With the carriage 30 fixed to the top end portion 106b of the outer sleeve 106, an activation of the motor 120 causes the carriage 30 to exert an upward force on the outer sleeve 106 to move the outer sleeve 106 upwardly relative to the inner shaft 104. As the outer sleeve 106 moves, the first and second pulleys 114a, 114b move therewith and relative to the third pulley 114c. In the fully extended position, as shown in FIG. 7, the slide 100 assumes a length substantially equal to the length of a conventional slide.

To lower the carriage 30 from the extended position, the motor 120 is activated to drive the belt 112 in the opposite direction. In the embodiment where the locking feature 118 fixes the carriage 30 to the top end portion 106b of the outer sleeve 106 of the slide 100, the downward force exerted on the carriage 30, via the belt 112, causes the outer sleeve 106 to retract relative to the inner shaft 104. The outer sleeve 106 may be retracted until the bottom end portion 106a of the outer sleeve 106 bottoms out on the bottom end portion 104a of the inner shaft 104. At this point, to further lower the carriage 30, the belt 112, via the motor 120, exerts a force great enough to unlock the carriage 30 from the top end portion 106a of the outer sleeve 106, whereby the carriage 30 descends along the track 102 of the outer sleeve 106 toward the descended position, as shown in FIGS. 3-5.

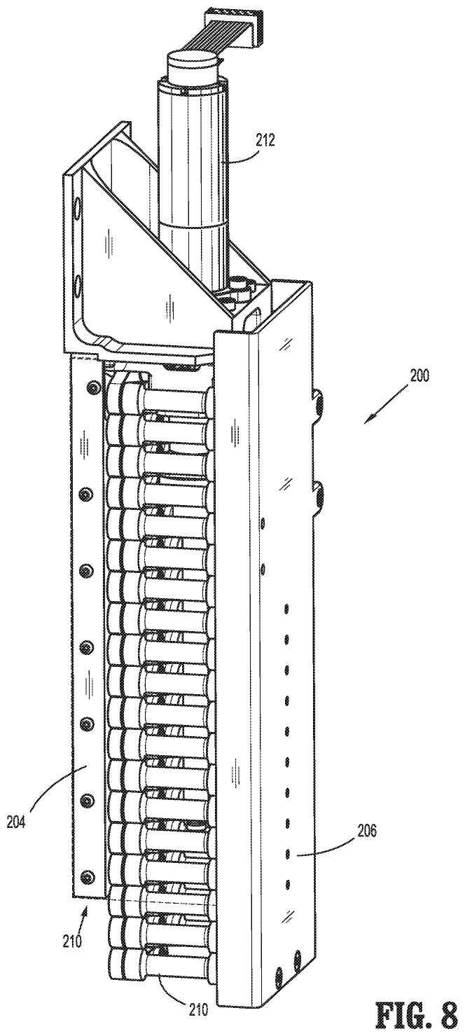

With reference to FIGS. 8-10, another embodiment of a slide 200 for use with the surgical robotic system of FIG. 1 is illustrated. Due to the similarities between the slide 200 of the present embodiment and the slide 100 described above, only those elements of the slide 200 deemed necessary to elucidate the differences from the slide 100 described above will be described in detail.

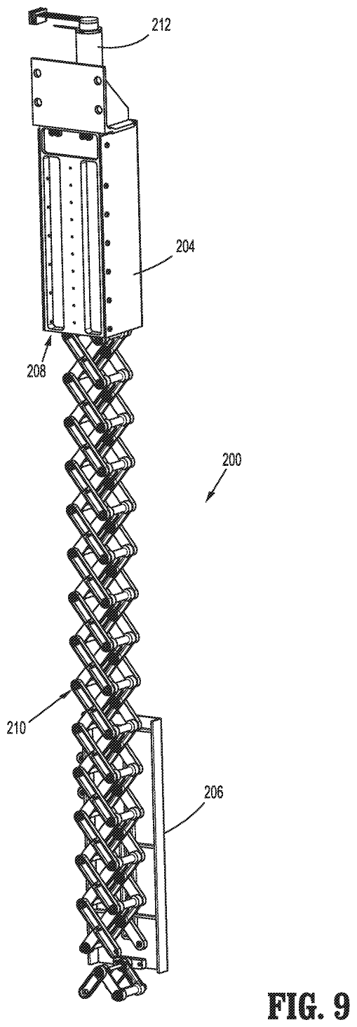

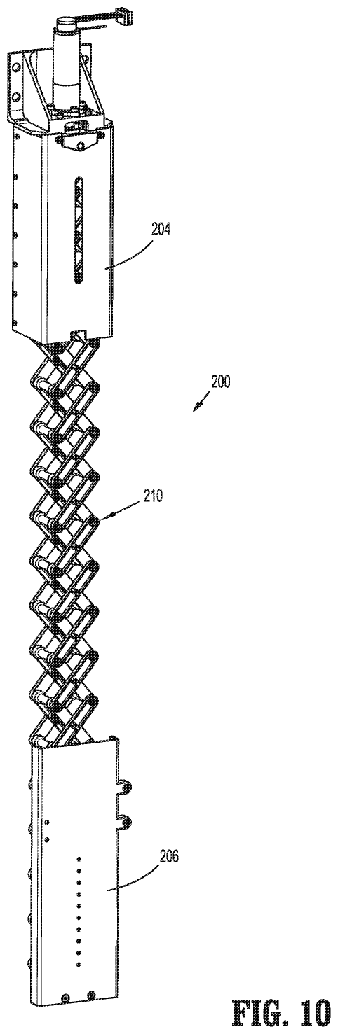

The slide 200 includes a main body portion 204 configured to be fixedly coupled to the end portion of the robotic arm 2 (FIG. 1), and a plate member 206 operably coupled to the main body portion 204. The main body portion 204 defines a cavity 208 therein having a retractable member 210 disposed therein. The retractable member 210 may be a scissors mechanism or pantograph that is transitionable from a retracted condition, as shown in FIG. 8, to an extended condition, as shown in FIGS. 9 and 10. A screw 212 may be coupled to the retractable member 210, such that a rotation of the screw 212 in a first direction causes the retractable member 210 to extend, whereas rotation of the screw 212 in a second direction causes the retractable member 210 to retract into the cavity 208 of the main body portion 204.

The plate member 206 is attached to a top end portion of the retractable member 210 and is configured to support the carriage 30 (FIGS. 2-7) of the instrument drive unit 20 thereon. As such, upon extending the retractable member 210, the plate member 206 is elevated relative to the main body portion 204 to raise the attached carriage 30 and/or instrument drive unit 20.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

1. A surgical robotic system, comprising:
an elongated slide defining a longitudinal axis and including:
an inner shaft; and
an outer sleeve disposed about the inner shaft and configured to move relative to the inner shaft along the longitudinal axis between a retracted position, in which the slide has a first length, and an extended position, in which the slide has a second length, greater than the first length; and
a carriage for supporting an instrument drive unit, wherein the carriage is coupled to the outer sleeve and movable relative to the outer sleeve along the longitudinal axis.

2. The surgical robotic system according to claim 1, wherein the carriage is movable along the outer sleeve between a descended position, in which the carriage is disposed adjacent a bottom end portion of the outer sleeve, and an ascended position, in which the carriage is disposed adjacent a top end portion of the outer sleeve.

3. The surgical robotic system according to claim 2, wherein the top end portion of the outer sleeve has a surface feature and the carriage is configured to engage the surface feature upon moving to the ascended position, such that further ascension of the carriage effects a movement of the outer sleeve toward the extended position.

4. The surgical robotic system according to claim 2, wherein the outer sleeve is configured to move toward the retracted position simultaneously with movement of the carriage toward the descended position.

5. The surgical robotic system according to claim 2, wherein the top end portion of the outer sleeve has a lock configured to selectively fix the carriage to the top end portion, such that a downward force exerted on the carriage is transferred to the outer sleeve to move the outer sleeve relative to the inner shaft from the extended position toward the retracted position.

6. The surgical robotic system according to claim 5, wherein the lock is configured to release the carriage upon the outer sleeve entering the retracted position, such that a further downward force exerted on the carriage causes the carriage to descend along the outer sleeve.

7. The surgical robotic system according to claim 1, further comprising a belt and pulley system operably coupled to the carriage for moving the carriage along the outer sleeve.

8. The surgical robotic system according to claim 7, wherein the belt and pulley system includes:
a pair of first and second pulleys coupled to the outer sleeve;
a third pulley coupled to the inner shaft; and
a belt operably coupled to each of the first, second and third pulleys, the carriage being fixed to the belt, such that movement of the belt drives a movement of the carriage along the outer sleeve.

9. The surgical robotic system according to claim 8, further comprising a motor operably coupled to the belt for driving the movement of the belt.

10. The surgical robotic system according to claim 8, wherein the first pulley is axially fixed to a top end portion of the outer sleeve, the second pulley is axially fixed to a bottom end portion of the outer sleeve, and the third pulley is axially fixed to a top end portion of the inner shaft.

11. The surgical robotic system according to claim 10, wherein the second pulley is disposed between the first and third pulleys, and wherein the first pulley is disposed a first distance from a vertical axis that extends through a center of the second pulley and parallel with the longitudinal axis of the slide, and the third pulley is disposed a second distance from the vertical axis, the second distance being less than the first distance.

12. The surgical robotic system according to claim 8, wherein the first and third pulleys are disposed adjacent one another when the outer sleeve is in the retracted position, and longitudinally spaced from one another when the sleeve is in the extended position.

13. The surgical robotic system according to claim 1, further comprising a robotic arm having the inner shaft coupled thereto.
